(19) United States
(12) Patent Application Publication
(10) Pub. No.: US 2009/0291110 A1
(43) Pub. Date: Nov. 26, 2009

(54) One swipe anti-bacteria disposable toilet seat wipe

(76) Inventors: Colbert Johnson, Homewood, IL (US); Velma Johnson, Homewood, IL (US)

Correspondence Address: Hiram R. Peacock, 12655 S. Lowe Ave., Chicago, IL, 60628, US

(21) Appl. No.: 12/152,628

(22) Filed: May 15, 2008

Related U.S. Application Data
(63) Continuation-in-part of application No. 11/699,623, filed on Jan. 30, 2007, now abandoned.
(60) Provisional application No. 60/763,057, filed on Jan. 30, 2006.

(51) Int. Cl. A01N 25/34; A01N 31/08; A01N 65/42; A01N 31/00; A01N 65/00; A01N 55/02; A61K 31/14; A01N 37/00; A01P 1/00

(52) U.S. Cl. 424/409; 514/731; 424/744; 514/724; 514/714; 424/725; 514/494; 424/771; 514/642; 514/557

(57) ABSTRACT

An improved anti-bacteria wipe for cleansing and drying toilet seats in public restrooms that is biodegradable and destroys germs and bacteria on contact. The wipe is comprised of four layers. The sealing first layer in conjunction with the third layer contains the moisture of the second layer. The pre-moistened second layer is infused with a quick drying anti-bacteria solution that kills germs and bacteria on contact. The third layer is an ultra thin biodegradable plastic to prevent seepage between the second and fourth layers. The fourth layer is a dry absorbent material used to remove any remaining moisture from the toilet seat. The wipe is sealed in an outer pouch to maximize shelf life. The biodegradable wipe can easily and safely be disposed of following use with reduced environmental impact.

### CROSS-REFERENCE TO RELATED APPLICATIONS

This application is a CIP of Ser. No. 11/699,623 filed Jan. 30, 2007, which claims priority of the PPA Ser. No. 60/763,057 filed on Jan. 30, 2006 by the present inventors, which are incorporated by reference.

### FIELD OF THE INVENTION

The present invention relates generally to a means for cleaning a toilet seat prior to use. More specifically, the invention provides an improved and more environmentally friendly toilet seat wipe for cleaning and drying a toilet seat prior to use.

### BACKGROUND OF THE INVENTION

As unpleasant as it may seem, public toilets are rife with germs and bacteria. According to a report filed by the American Medical Association (AMA), touching a toilet seat just once can leave enough germs and bacteria on the skin, that hands must be washed at least five to six times consecutively, just to get rid of the majority of these harmful agents. Easily thriving on hands, these germs and bacteria are also easily deposited onto the legs and buttocks. Realistically, few people, if anyone, would have time or even consider showering five or six times after using the restroom. Most public toilets are a breeding ground for microscopic fecal coli form, a potentially deadly form of the E. Coli virus. In fact, the AMA also reports that as many as 25 percent of people leave the restroom with traces of fecal coliform (usually E. Coli) on their bodies. Furthermore, it has been found that the common cold and influenza are easily transmitted from one person to the next, via the public toilet. While anyone can be exposed to illness from contact with a contaminated toilet seat, elderly persons, those suffering with immune deficiency diseases, chemotherapy patients, pregnant women, young children, and people with diabetes are particularly susceptible to these germs and bacteria.

Clearly no matter how well a rest room appears to be maintained, it can nonetheless be a breeding ground for infectious matter. For the millions of those who must share toilet facilities at work, and for the countless others who must use the restroom when shopping, dining out, going to a movie, or in any other public place, using a public toilet can be an unsettling prospect. Perhaps this is why so many people improvise; covering the toilet seat with paper towels, tissue or toilet paper; all in attempts to avoid contact with the germs and bacteria that are left behind by other people. An awkward solution at best, covering the toilet seat in this manner most often leads to clogged plumbing and excessive amounts of waste paper on the restroom floor.

As a solution to this problem, many public restrooms have begun providing ultra-thin disposable toilet seat covers inside toilet stalls to help users with their apprehension of coming into direct contact with the toilet seat. These seat covers are typically made of a paper material and are in no way preventive of the spread of microorganisms, especially if they come into contact with liquids whether splashed or sprayed from within or around the toilet. The seat covers also do not address the issue of destroying bacteria and microorganisms that are present on the toilet seat.

Most anti-bacteria wipes act as a disinfectant which consists of an agent that does not ordinarily have the necessary ingredients to remove most or all of the microorganisms that may be found on a soiled toilet seat on contact.

Various pre-moistened disposable toilet seat cleaning products are known in the art. Typical of such wipes and wiping devices are the ones shown in U.S. Pat. Nos. 5,025,524; 4,601,081; 4,575,891; and 4,998,984, all using various methods for cleaning a toilet seat. There remains a need for an improved wipe capable of destroying bacteria and drying a public toilet seat to allow a user to use a public restroom without apprehension.

### OBJECTS AND ADVANTAGES

The object of the present invention is to overcome the aforementioned disadvantages of prior art wipes and provide an anti-bacteria wipe that is affordable, easily portable, environmentally friendly, and easy to manufacture.

It is a further object of the invention to provide a wipe that effectively eliminates the spread of germs and bacteria between users.

It is a further object of this invention to provide a wipe that is biodegradable, and can be easily disposed of without the indefinite lifespan of traditional plastic products.

It is a further object of this invention to provide a wipe that would greatly reduce the threat of communicable diseases, such as the common cold, flu, or in more serious instances, the E. Coli virus, from infiltrating the body's immune system.

### SUMMARY OF THE INVENTION

The One Swipe Anti-bacteria Disposable Toilet Seat Wipe is specifically designed to act as a cleansing and drying device to remove moisture from the toilet seat prior to use, as well as killing most germs on contact. The One Swipe Anti-bacteria Disposable Toilet Seat Wipe is a two sided wipe comprised of four layers of biodegradable materials, where the first and third layers are both comprised of a biodegradable plastic. The first layer in conjunction with the third layer provides a removable seal for the liquid contained in the wet second layer. The sealing of the first and third layers around the wet second layer prevents leakage of the anti-bacteria solution prior to use. The biodegradable plastic of the first and third layers also keep the pre-moistened anti-bacteria solution in the absorbent second layer separate from the absorbent drying pad of the fourth layer. The anti-bacteria solution infused into the second layer is comprised of a solution capable of neutralizing endospores and pathogens, E. Coli and influenza, bodily fluids including blood, or even HIV and the AIDS virus.

### DETAILED DESCRIPTION

The present invention provides a practical solution to the aforementioned challenges. The One Swipe Anti-bacteria Disposable Toilet Seat Wipe is a biodegradable wipe, designed specifically to be effortlessly glided across the surface of a toilet seat, killing germs and bacteria on contact.

As seen in FIG. 1 and FIG. 2, each One Swipe Anti-bacteria Disposable Toilet Seat Wipe 10 is constructed of a layer of square or rectangular biodegradable and disposable sanitary material contained between the first and third layers of biodegradable plastic which act as a sealed container surrounding the wet second layer used for wiping the toilet seat. The wet layer 14 of each One Swipe Anti-bacteria Disposable Toilet Seat Wipe is infused with a hypoallergenic anti-bacteria solution, formulated to kill germs and bacteria on contact. The wet layer 14 can be composed of any suitable material including synthetic fibers, natural fibers, and unwoven biodegradable material or paper such as wet strength tissue or dry laid paper. The dry layer 18 of the wipe is a dry absorbent pad for use in drying the toilet seat after the solution has been applied. The wet second layer 14 is separated from the dry fourth layer 18 by a liquid impermeable third layer 16 comprised of an ultra thin biodegradable plastic. The ultra thin biodegradable plastic barrier layer 16 prevents the solution from seeping through from the wet layer 14 to the dry layer 18. The first layer 12 in conjunction with the third layer 16 removably acts as a sealing containment for the wet layer 14 of the wipe. The One Swipe Anti-bacteria Disposable Toilet Seat Wipe 10 can be produced in fragranced, fragrance free, as well as with alcohol or alcohol free varieties. FIG. 3 illustrates the One Swipe Anti-bacteria Disposable Toilet Seat Wipe 10 individually wrapped in a sealed plastic pouch 20, the would be easily accessed from this packaging by way of a handy pull tab 22, which runs along the upper length of the pouch. The front of the pouch carries the One Swipe Anti-bacteria Disposable Toilet Seat Wipe logo, while instructions for use are imprinted on the back of the pouch. The One Swipe Anti-bacteria Disposable Toilet Seat Wipe 10 could also be sold in multi-package containers, ideal for household use. Alternately, the One Swipe Anti-bacteria Disposable Toilet Seat Wipe 10 could be dispensed in vending machines such as those found in restrooms in public establishments.

Use of the One Swipe Anti-bacteria Disposable Toilet Seat Wipe 10 is very simple and straight forward. First, opening the One Swipe Anti-bacteria Disposable Toilet Seat Wipe pouch 20 via the pull tab 22, the user would remove the wipe 10 from the storage pouch 20. Next the user would remove the first biodegradable plastic layer 12 to expose the moistened second layer 14. The user would then simply swipe the pre-moistened second layer 14 of the One Swipe Anti-bacteria Disposable Toilet Seat Wipe 10 across the surface of the toilet seat, enabling the anti-bacteria solution to kill germs and bacteria on contact. To ensure maximum dryness of the toilet seat before use, the user can turn the wipe over so that the dry fourth layer 18 faces down, and dry the surface of the toilet seat with the dry side of the wipe. After drying the seat, the user could then safely use the freshly cleansed facilities. The soiled wipe 10 can be returned into the storage pouch 20, and the storage pouch 20 placed in the nearest trash receptacle.

### BRIEF DESCRIPTION OF THE DRAWINGS

This invention, together with other objects, features, aspects and advantages thereof, will be more closely understood from the following descriptions, considered in conjunction with the accompanying drawings.

FIG. 1 is an exploded view of the One Swipe Anti-bacteria Disposable Toilet Seat Wipe.

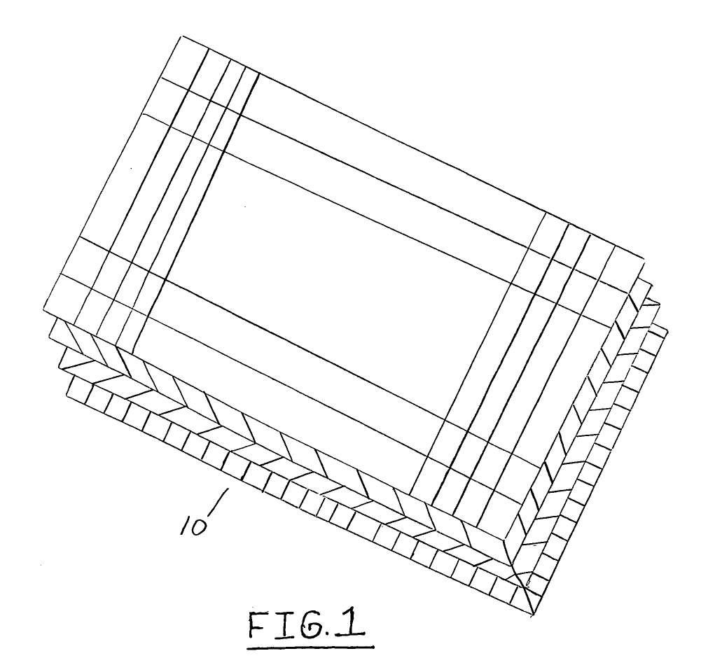

FIG. 2 is a side view of the One Swipe Anti-bacteria Disposable Toilet Seat Wipe.

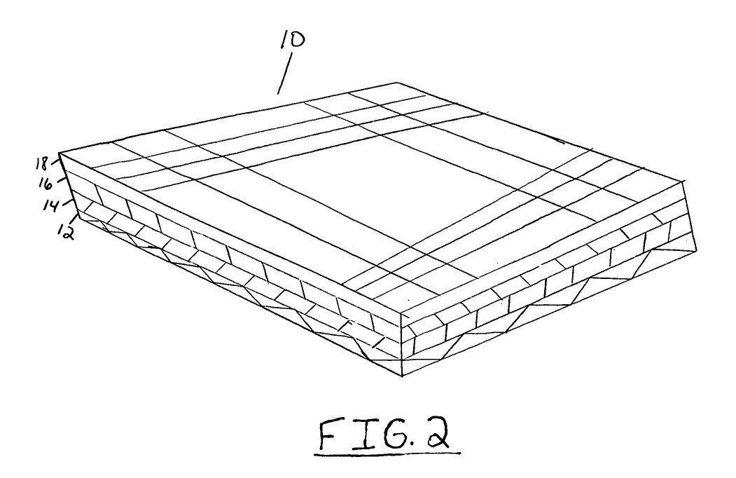

FIG. 3 is a perspective top view of the sealed storage pouch that contains the One Swipe Anti-bacteria Disposable Toilet Seat Wipe

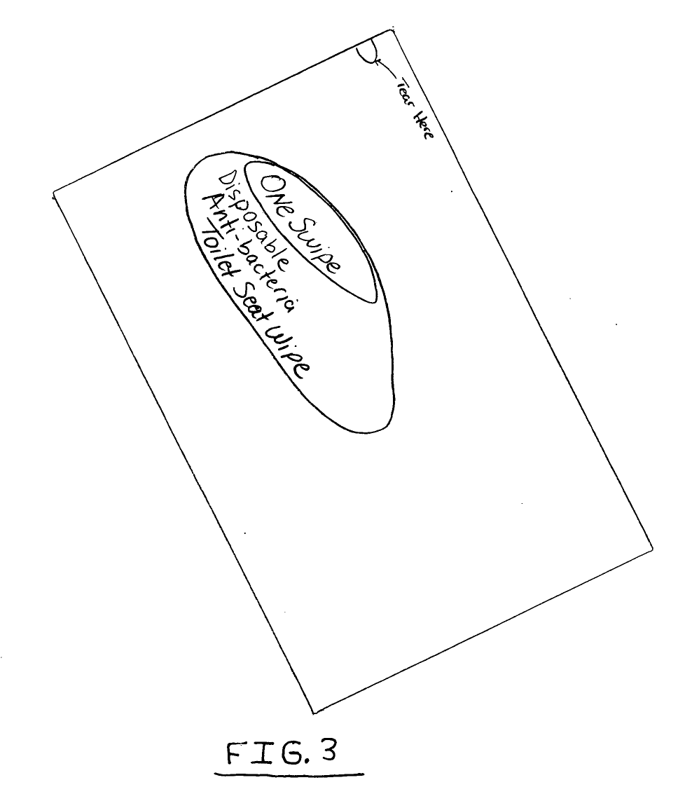

### DRAWINGS—REFERENCE NUMERALS

10 One Swipe Anti-bacteria Disposable Toilet Seat Wipe

12 First biodegradable plastic sealing layer

14 Anti-bacteria solution infused wiping second layer

16 Biodegradable plastic sealing third layer

18 Biodegradable drying fourth layer

20 Sealed storage pouch

22 Pull tab

### PREFERRED EMBODIMENTS

In a preferred embodiment the One Swipe Anti-bacteria Disposable Toilet Seat Wipe is approximately 3″×3″ square. The non-woven fiber of the wet layer is absorbent yet capable of releasing the quick drying anti-bacteria solution upon contact with the toilet seat. The biodegradable plastic first and third layers prevent seepage of the solution onto the dry fourth layer of absorbent pad that is used to complete the drying of the toilet seat.

### CONCLUSION, RAMIFICATIONS AND SCOPE

Accordingly, the reader will see that the One Swipe Anti-bacteria Disposable Toilet Seat Wipe provides an effective portable and environmentally friendly wipe for users of public restroom facilities. Some of the additional advantages of the One Swipe Anti-bacteria Disposable Toilet Seat Wipe are that:
1. It offers a simple and effective means of preventing the spread of communicable diseases;
2. It is extremely portable and can be used to cleanse other public facilities including public telephones, restroom door handles, and a variety of other breeding grounds;
3. Its small size and biodegradability prevent undue strain on the waste disposal system and the environment.

Although the description above contains much specificity, it should not be construed as limiting the scope of the invention, but as merely providing illustrations of some of the presently preferred embodiments of this invention. For example, other types of anti-bacteria agents can replace the existing ones; and additional restorative and natural emollients could be added. Additionally the wipe can be manufactured in a wide variety of sizes including wipes as small as 2″×1″ or approximately 3″×7″, which is large enough to cover the entire hand of a user. Thus the scope of the invention should be determined by the appended claims and their legal equivalents, rather than by the examples given.

What is claimed is:

1. A one swipe anti-bacteria disposable toilet seat wipe comprising:
a removable biodegradable plastic first layer;
an absorbent second layer infused with an anti-bacteria solution;
a biodegradable plastic third layer, being connected to the removable first layer to form a sealed container for said second layer, having a top side and a bottom side, wherein the top side is peripherally attached to said second layer;
a substantially dry and absorbent fourth layer that is attached to the bottom side of said third layer.

2. The one swipe anti-bacteria disposable toilet seat wipe of claim 1 wherein the solution infused in the second layer is capable of cleansing the toilet seat on contact.

3. The one swipe anti-bacteria disposable toilet seat wipe of claim 2 wherein the wipe is substantially biodegradable within 6 months of placement in a landfill.

4. The one swipe anti-bacteria disposable toilet seat wipe of claim 3 further comprising a sealable biodegradable plastic pouch to enclose the wipe;
said sealable pouch having a pull tab to break the seal on the pouch.

5. The one swipe anti-bacteria disposable toilet seat wipe of claim 2 wherein the solution infused in the second layer is contains an aqueous alcoholic composition to aid in quick drying.

4. The one swipe anti-bacteria disposable toilet seat wipe of claim 2 wherein the anti-bacteria solution is selected from the group comprising: a germicide, a sterilizer, an aqueous alcoholic composition, an emollient, a fragrance, and combinations thereof.

5. The one swipe anti-bacteria disposable toilet seat wipe of claim 1 wherein the anti-bacteria solution is selected from the group comprising: a phenol compound, aloe vera, an aqueous alcoholic composition, hydro-peroxide, leptospermum scoparium, kunzea ericoides, and combinations thereof.

6. The one swipe anti-bacteria disposable toilet seat wipe of claim 1 wherein the anti-bacteria solution is selected from the group comprising: purified or distilled water, liquid soap, zinc pyrithione, black walnut green hull extract, olive leaf, aloe vera, leptospermum scoparium, kunzea ericoides, and combinations thereof.

7. A wiping device for disinfecting and eliminating bacteria from toilet seats on contact comprising:
a first layer comprised of a biodegradable plastic;
a second layer having a top side and a bottom side;
said second layer including an infusion of a liquid anti-bacteria means formulated to kill germs and bacteria on contact;
said second layer being composed of a biodegradable sanitary paper;
a third layer having a top side and a bottom side;
said top side of the third layer being attached to the bottom side of said second layer;
said third layer being composed of a biodegradable plastic to prevent moisture seepage between the layers;
a fourth layer having a top side and a bottom side;
said fourth layer being composed of substantially dry absorbent biodegradable material;
said top side of the fourth layer being attached to the bottom side of the third layer.

8. The wiping device of claim 7 further comprising a sealable plastic pouch to enclose the wipe;
said sealable pouch having a pull tab to break the seal on the pouch.

9. The wiping device of claim 8 wherein said sealable plastic pouch is biodegradable.

10. The one swipe anti-bacteria wipe of claim 1, further comprising a sealable plastic pouch to enclose the wipe;
said sealable pouch having a pull tab to break the seal on the pouch.

11. A one swipe anti-bacteria disposable toilet seat wipe comprising:
a sealing top layer;
an absorbent wet layer infused with an anti-bacteria solution;
a dividing seal layer, being substantially impervious to the solution of said wet layer and having a top side and a bottom side, wherein the top side is attached to said wet layer;
a substantially dry and absorbent layer that is attached to the bottom side of said dividing seal layer.

12. The one swipe anti-bacteria disposable toilet seat wipe of claim 11 wherein said anti-bacteria solution is comprised of liquids selected from the group of: purified or distilled water, liquid soap, zinc pyrithione, black walnut green hull extract, olive leaf, aloe vera, leptospermum scoparium, kunzea ericoides, and combinations thereof.

13. The one swipe anti-bacteria disposable toilet seat wipe of claim 11 wherein said anti-bacteria solution is comprised of liquids selected from the group of: purified or distilled water, benzalkonium chloride, potassium sorbate, alcohol 40, liquid soap and combinations thereof.
